(19) United States
(12) Patent Application Publication
(10) Pub. No.: US 2007/0259058 A1
(43) Pub. Date: Nov. 8, 2007

(54) Morinda herbal extracts and compositions thereof

(76) Inventor: Rulin Xiu, Pahoa, HI (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/417,223

(22) Filed: May 4, 2006

(51) Int. Cl. A61K 36/746

(52) U.S. Cl. 424/769; 424/777

(57) ABSTRACT

The present invention provides herbal extracts, and methods of making them, utilizing a Morinda solution as an extraction medium. It has been found herein that herbal extraction procedures that use Morinda solutions as a solvent produce an effective herbal extract product with enhanced and synergistic properties.

Morinda citrifolia is a medicinal plant that has been reported to have a broad range of therapeutic effects, including antibacterial, antifungal, antitumor, analgesic, hypotensive, anti-inflammatory, and immune enhancing effects. The plant fruit and other plant parts have been used to treat various disorders and diseases, including, arthritis, diabetes, high blood pressure, muscle disorders, headaches, heart disease, cancer, gastric ulcers, mental disorders (including depression and dementia), digestive disorders, wounds (e.g., to promote healing), and arteriosclerosis. Morinda has been reported to contain a large number of biologically-active agents, including, e.g., scopoletin, octoanoic acid, potassium, Vitamin C, terpenoids, alkaloids, anthraquinones, beta-sitosterol, carotene, Vitamin A, flavone glycosides, linoleic acid, Alizarin, acubin, L-asperuloside, caproic acid, caprylic acid, ursolic acid, rutin, asperulosic acid, proxeronine, and xeronine. See, e.g., Mian-Ying et al., Acta Pharmacol. Sin., 12:1127-1141, 2002.

The present invention provides herbal extracts and methods of making them, especially extracts that have been prepared utilizing a Morinda solution as an extraction medium. It has been found herein that herbal extraction procedures that use Morinda solution as a solvent produce an effective herbal product with enhanced and synergistic properties.

The present invention relates to the use of Morinda citrifolia (commonly known as the Indian Mulberry plant) as a solvent in any suitable herbal extraction method. Morinda is an evergreen shrub, or a small or medium sized tree that reaches from about three to ten meters in height at maturity. It is also known commercially as “noni.” It is a member of the Rubiaceae (coffee family), and the subfamily Rubioideae. Morinda grows in tropical coastal regions around the world. The plant leaves are opposite pinnately veined, and glossy. The leaves are broadly elliptic to oblong, pointed at both ends, ten to thirty centimeters in length and five to fifteen centimeters wide. The Morinda flowers are small, white, three to five-lobed, tubular, fragrant, and about one and one-quarter centimeters long. The flowers develop into compound fruits composed of many small drupes fused into an ovoid, ellipsoid or roundish, lumpy body, five to ten centimeters long, five to seven centimeters thick, with waxy, white or greenish-white or yellowish, semi-translucent skin. The fruit contains “eye” on its surface, similar to a potato. The fruit is juicy, bitter, dull-yellow or yellowish-white, and contains numerous red-brown, hard oblong-triangular, winged, two-celled stones, each containing about four seeds. When fully ripe, the fruit has a pronounced disagreeable odor like rancid cheese. Morinda can be propagated from seeds, stem, or root-cuttings. See, e.g., worldwide web at traditionaltree.org, Species Profiles for Pacific Island Agroforestry, S. C. Nelson, April 2006, ver. 4; U.S. Pat. No. 7,018,662.

A Morinda solution in accordance with the present invention comprises the juice or extract of a Morinda plant part. Plant parts include, e.g., seeds, leaves, root, fruit, branch, bark, stem, flower, and mixtures thereof. The solution can be 100% aqueous and/or comprise alcohol. A Morinda fruit solution is a juice of the Morinda fruit. Ground or powdered leaves, root, seeds, branch, bark, stem, flower, or oil extracts thereof, can be optionally added to the Morinda fruit solution. A solution can comprise, e.g., at least about 20%, 30%, 40%, 50%, 60%, 70%, 80%, 90%, 100% Morinda juice (fresh or fermented). A Morinda fruit solution can also comprise, e.g., 5 parts Morinda juice (unadulterated) to 1 part of other ingredients, such as water or other juices. A fruit solution can be utilized after being concentrated (e.g., after boiling), where it is concentrated by, e.g., 1×, 2×, 5×, 10×, 20×, 25×, 50×, 100×, etc. See, e.g., U.S. Pat. No. 6,855,354 for a freeze concentration method as applied to Morinda. Concentration processes for fruit juices can involve, e.g., evaporation, distillation, prevaporation, crystallization, freezing, pressing, spray dry, lyphilization, etc.

Processed Morinda juice can be prepared routinely, e.g., by separating the seeds and peels from the juice and pulp of a ripened Morinda fruit (e.g., fresh squeezed using a mechanical device), and optionally removing the pulp from the juice by filtration or centrifugation. For example, a plate filtration unit with cellulose filters (e.g., from 8-20 microns) can be utilized to deplete the juice of pulp and other particulate materials.

The fruit can be harvested when it is at least one inch (two to three centimeters) and up to twelve inches (twenty-four to thirty-six centimeters) in diameter. The fruit preferably has a color ranging from a dark-green through a yellow-green up to a white color, and gradations of color in between. The fruit can be ripened or aged, e.g., from 1-14 days. When ready for further processing the fruit is typically light in color, from a light green, light yellow, white or translucent color. The juice product can be heated and pasteurized at a temperature of, e.g., from about 83° C.-100° C.

Morinda solution can also be a fermented product. For example, ripe fruits can be pulped, and then placed into large fermentation containers, optionally with added water, enzymes, sugars, and other conventional components. The juice can separate naturally from the fruit pulp, and ferment naturally via a microbial process (e.g., bacteria and yeast). Fermentation periods can be varied, e.g., depending on the degree of alcoholic content desired, e.g., 30 days, 60 days, 3 months, 6 months, etc. The fermented product, with a low pH, can be stored without pasteurization and utilized in the herbal extraction process. See, also, Newton, Proceedings of the 2002 Hawai'I Noni Conference, Pages 29-32, 2002.

Morinda juice (fresh-squeezed; fermented) can be further processed, e.g., lyophilized; concentrated (e.g., using evaporation technologies). The fruit, itself, can be dehydrated, milled, and formed into a powder, which can then be re-processed to form a Morinda solution. Other Morinda plant parts can be processed similarly. See, e.g., U.S. Pat. No. 5,288,491 which describes a method of making a Morinda citrifolia powder.

The present invention provides methods of preparing an herbal extract, comprising, e.g., contacting a Morinda solution (e.g., comprising fruit, seed, leaves, root, branch, bark, stem, flower, etc.) with a solid herbal material under conditions which are effective to extract soluble compounds from said herbal material into said Morinda fruit solution to form a Morinda extract of said herbal material.

A Morinda fruit solution can be prepared routinely, e.g., as described above. Extraction can be accomplished using any suitable extraction method. The present invention is not limited to how the extraction is achieved. For example, extraction can be effected under any suitable condition, including, but not limited to, e.g., pressure (both low and high), temperature (e.g., boiling); under gas (including oxygen; inert gases, such as nitrogen; carbon dioxide); under vacuum; etc.

The herbal material is referred to as solid to generally indicate that it contains a particulate and/or insoluble component. However, the solid material does not have to comprise 100% insoluble parts. Generally, when an herb is to be extracted, the cell walls of the plant are opened to allow solvent access to the cellular content. This can be accomplished in any effective method, e.g., using milling machinery (e.g., that grind the material through friction); filter presses; grinding mills; cam grinding; etc. Thus, the material can be in any form that is suitable for the extraction process, including, milled, pulverized, powdered, ground, mashed (e.g., where it could be utilized as a pulp comprising aqueous and insoluble fiber materials), sliced, minced, chopped, etc. Solid materials can also comprise oils, e.g., where plant materials have been pressed or otherwise treated to at least partially extract biologically active oils.

The herbal material can be contacted with the juice solution, and stored for any suitable period of time to effect the extraction process, e.g., 1 day, 2, days, 5 days, 1 week, 1 month, 6 months etc. During this period, soluble components of the herb can diffuse or be released into the Morinda solvent.

The extraction step can also be performed with a fermented Morinda juice solution comprising alcohol. Such alcohol content can be, e.g., from about, or to, 0.1%, 0.2%, 0.3%, 0.4%, 0.5%, 0.6%, 0.8%, 0.9%, 1%, 1.5%, 2%, 2.5%, 3%, 4%, 5%, 6%, 7%, 8%, 9%, 10%, 11%, 12%, 13%, 14%, 15%, 20%, 25%, etc. Preferred range are from about 3% to about 15% alcohol content. The herbal material can be contacted with the fermented juice solution, and stored for any suitable period of time to effect the extraction process, e.g., 1 day, 2, days, 5 days, 1 week, 1 month, 6 months etc. During this period, soluble components of the herb can diffuse or be released into the Morinda solvent. Alternatively, the herbal material can be added to non-fermented Morinda solution, and then stored under conditions effective to cause fermentation. For example, the solution can be stored in a fermentation container at a desired temperature, e.g., from 25-60° C., for 1 day to 6 months. Optionally, yeast and/or bacteria can be added to initiate or augment the fermentation process.

An extraction process according to the present invention can involve contacting one or more herbal materials in a particular ratio with a Morinda solution; extracting the materials under reduced pressure; and further concentrating by constant boiling. The resulting extract can optionally be subjected to filtration or centrifugation to remove particulate materials. See, e.g., U.S. Pat. No. 5,910,307. An extraction process is also described in U.S. Pat. No. 5,466,454 which describes boiling herbs in a solvent to produce an herbal extract concentrate. A percolation process can also be utilized to produce herbal extracts. See, e.g., U.S. Pat. No. 6,555,074. Herbal extracts can be concentrated by any desired amount, e.g., concentrated by, e.g., 1×, 2×, 5×, 10×, 20×, 25×, 50×, 100×, 200×, 500×, 1000×, etc.

The present invention also relates to an herbal extract solution, comprising an extracted herbal material present in Morinda solution. By “present in Morinda solution,” it is meant that the herbal materials have been extracted with Morinda as a functional solvent. The herbal extract solution can be prepared as indicated above, as well as according to any suitable extraction procedure using a Morinda solvent to solubilize the components of the herb. An herbal extract can contain a desired parts by weight of extracted material, e.g. at least 0.5, 1, 2, 5, 10, 20, 25, 50, 75, 100, 200, 500, 1000, or more parts, of the total extract weight. For example, if 10 grams of herbal material were extracted with 10 mls of Morinda solvent, and this was then concentrated into 1 ml, then final amount would be about 10 gm/1 gm or 10 parts herbal material per 1 part Morinda solution. The parts of herbal material represents the components which were extracted from the total herbal material utilized in the extraction process. A extract contains about 5 parts by weight of extracted material.

An herbal material of the present invention can be any plant material that is used for medicinal or nutritive purposes. Examples, include without limitation, ginseng, cibotium barometz, dryopteris crassirhizoma, morus alba, lycium chinense, picrorhiza, rehmannia glutinosa, cistanche, deserticola, lonicera, platycodon grandiflorus, atractylodes, etc. Fresh and/or dried materials can be utilized. Other examples, include, Sophora flavescens Ait, Polygonum multiflorum Thunb, Valeriana pseudofficinalis, Panax notoginseng root and leaf, Morinda officinalis How root, Albizzia julibrissin durazz bark and flower, Cyperus rotundus L. root, Tribulus terrestris fruit, Epimedium brevicomum root, Acanthopanax senticosus Harms, Caulis Spatholobi, Daikon (Radish) seed, Eucommia ulmoides oliv, Astragalus membranaceus, Rhodiola Crenulata, Cibotium barometz, Cyathula officianalis Kuan, Cynanchum paniculatum root, Angelica biserrata root, Geranium wilfordii maxim, Curculigo orchioides gaertn, Cnidium monnieri fruit, Erigerm breviscapus Hand.-Mazz, Cassia tora, Chrysanthemum morifolium Ramat., Ginkgo Biloba L, Chrysanthemum morifolium Ramat., Bitter melon leaf and fruit, Cyperus rotundus L. root, Semen canavaliae ensiformis, Syzygium aromaticum Merr, Diospyros kaki thunb, etc.

The amount of material which is contacted with the Morinda solution can be varied as desired. For example, the ratio of solution to herbal material can be, e.g., from about 0.1:100; from about 1:100; from about 1:50; from about 1:10; from 1:5; from about 1:2; from about 1:1; from about 2:1; from about 5:1; from about 10:1; from about 20:1; from about 50:1; from about 100:1; etc.

The herbal extracts can be administered in any form by any effective route, including, e.g., oral, parenteral, enteral, intraperitoneal, topical, transdermal (e.g., using any standard patch), ophthalmic, nasally, local, non-oral, such as aerosal, spray, inhalation, subcutaneous, intravenous, intramuscular, buccal, sublingual, rectal, vaginal, intra-arterial, and intrathecal, etc.

Without further elaboration, it is believed that one skilled in the art can, using the preceding description, utilize the present invention to its fullest extent. The specific embodiments are to be construed as merely illustrative, and not limitative of the remainder of the disclosure in any way whatsoever.

The entire disclosures of all applications, patents and publications, cited herein are incorporated by reference herein in their entirety.

From the foregoing description, one skilled in the art can easily ascertain the essential characteristics of this invention and, without departing from the spirit and scope thereof, can make various changes and modifications of the invention to adapt it to various usages and conditions.

What we claim:

1. A method of preparing an herbal extract, comprising:
contacting a Morinda fruit solution comprising a solid herbal material under conditions which are effective to extract soluble compounds from said herbal material into said Morinda fruit solution to form a Morinda extract of said herbal material.

2. A method of claim 1, further comprising separating said solid herbal material from said Morinda solution.

3. A method of claim 1, wherein said Morinda solution is an aqueous solution.

4. A method of claim 1, wherein said Morinda solution is an alcoholic solution.

5. A method of claim 1, further comprising concentrating said extract of said herbal material.

6. A method of claim 1, wherein said conditions effective to extract soluble compounds from said herbal material comprises concentrating said Morinda solution under reduced pressure.

7. A method of claim 1, wherein said conditions effective to extract soluble compounds from said herbal material comprises boiling said Morinda solution.

8. A method of claim 1, wherein said conditions effective to extract soluble compounds from said herbal material comprises storing said solution at a temperature of 25°-60° C. for 1-30 days under conditions effective to produce fermentation.

9. A method of claim 1, further comprising lyophilizing said extract.

10. A method of claim 1, wherein the ratio of Morinda solution to herbal material is from 1:1 to 10:1.

11. A method of claim 1, wherein said herbal matter is fresh herb.

12. A method of claim 1, wherein said herbal matter is dried herb.

13. A method of claim 1, wherein said Morinda solution comprises an extract of Morinda seeds, leaves, branch, bark, stem, flower, and/or root.

14. An herbal extract solution, comprising an extract herbal material present in Morinda solution.

15. An herbal extract of claim 1, wherein said extract comprises an extract of herbal material which is at least 5:1 parts of herbal material per part of herbal extract.
